(19) United States
(12) Patent Application Publication
(10) Pub. No.: US 2022/0192484 A1
(43) Pub. Date: Jun. 23, 2022

(54) METHOD FOR DIFFERENTIATING BETWEEN PAPILLEDEMA AND PSEUDOPAPILLEDEMA IN A SUBJECT

(71) Applicant: Vanderbilt University, Nashville, TN (US)

(72) Inventors: Sean P. Donahue, Nashville, TN (US); Alexis Flowers, Nashville, TN (US); Qingxia Chen, Nashville, TN (US); Yuhan Liu, Nashville, TN (US); Reid Longmuir, Nashville, TN (US)

(21) Appl. No.: 17/554,376

(22) Filed: Dec. 17, 2021

Related U.S. Application Data
(60) Provisional application No. 63/128,275, filed on Dec. 21, 2020.

(51) Int. Cl. A61B 3/10; A61B 3/12

(52) U.S. Cl. CPC A61B 3/1005; A61B 3/1225; A61B 3/102

(57) ABSTRACT

A method for differentiating between papilledema and pseudopapilledema involves identifying a subject with an elevated optic nerve, and detecting in the subject a retinal nerve fiber layer (RNFL) thickness at each of twelve clock-hour positions using optical coherence tomography (OCT).

### RELATED APPLICATIONS

This application claims priority from U.S. Provisional Application Ser. No. 63/128,275 filed Dec. 21, 2020, the entire disclosure of which is incorporated herein by this reference.

### TECHNICAL FIELD

The presently-disclosed subject matter generally relates to clinical identification of papilledema and pseudopapilledema. In particular, certain embodiments of the presently-disclosed subject matter relate to differentiating between papilledema and pseudopapilledema in a subject having an elevated optic nerve.

### INTRODUCTION

A longstanding dilemma for the ophthalmologist is determining whether an elevated optic nerve represents papilledema or pseudopapilledema. Papilledema refers to swelling of the optic nerve due to increased intracranial pressure, and indicates a need for treatment. Sometimes, the optic nerve is elevated and appears swollen, but it is due to other reasons. This condition is referred to as pseudopapilledema and does not require treatment. It can be very difficult clinically to distinguish the two.

Previous studies have analyzed different imaging modalities, including ultrasonography, optical coherence tomography (OCT), and OCT angiography for use in supplementing the clinical exam to help differentiate papilledema verses pseudopapilledema. Previous studies looking at OCT have compared quadrant measurements, features of the subretinal fluid space, and Bruch's membrane opening. However, reports from such previous studies have found that quantitative OCT cannot distinguish between papilledema or pseudopapilledema. There is a need in the art for distinguishing between papilledema or pseudopapilledema in a subject with an elevated optic nerve.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes an Optic Disc Edema Index (ODEI), which can be used to separate papilledema from pseudopapilledema with high sensitivity and specificity. The ODEI is useful in both adult and pediatric populations.

In some embodiments, the presently-disclosed subject matter includes a method for differentiating between papilledema and pseudopapilledema involves identifying a subject with an elevated optic nerve, and detecting in the subject a retinal nerve fiber layer (RNFL) thickness at each of twelve clock-hour positions using optical coherence tomography (OCT). In this regard, reference is made to FIG. 1, which illustrates 30 degree portions in a clock hour distribution relative to the temporal (T) and nasal (N) retinal positions.

In some embodiments, the presently-disclosed subject matter includes a method for determining a magnitude and variability of RNFL thickness of an elevated optic nerve in a subject, which involves detecting in the subject a RNFL thickness for each of twelve clock-hour positions (as depicted in FIG. 1), calculating a magnitude value (M), calculating a variability value (V), and calculating a magnitude-variability value (X).

In some embodiments, the presently-disclosed subject matter includes a method for differentiating between papilledema and pseudopapilledema, which involves identifying a subject with an elevated optic nerve, detecting in the subject a retinal nerve fiber layer (RNFL) thickness at each of twelve clock-hour positions using optical coherence tomography (OCT). In some embodiments, the method includes identifying the subject as having pseudopapilledema when Xis less than about 13.2, wherein X=(B₁*M)+(B₂*V). In some embodiments, the method includes identifying the subject as having papilledema when X is greater than about 13.2, wherein X=(B₁*M)+(B₂*V). In some embodiments, the method includes identifying the subject as having papilledema when X is greater than about 14.8, wherein X=(B₁*M)+(B₂*V).

In some embodiments, the magnitude value (M) is (Y₁+Y₂+Y₃+Y₄+Y₅+Y₆+Y₇+Y₈+Y₉+Y₁₀+Y₁₁+Y₁₂)/12, wherein Yₙ is the RNFL thickness for the n clock hour position.

In some embodiments, the variability value (V) is (Y₁−Y₁₂)+(Y₂−Y₁)+(Y₃−Y₂)+(Y₄−Y₃)+(Y₅−Y₄)+(Y₆−Y₅)+(Y₇−Y₆)+(Y₈−Y₇)+(Y₉−Y₈)+(Y₁₀−Y₉)+(Y₁₁−Y₁₀)+(Y₁₂−Y₁₁), wherein Yₙ is the BNFL thickness for the n clock hour position.

In some embodiments, the magnitude-variability value (X), where X=(B₁*M)+(B₂*V), B₁ is a magnitude coefficient, and B₂ is a variability coefficient;

In some embodiments, methods as disclosed herein B₁ is about 0.08-0.12. In some embodiments, methods as disclosed herein B₁ is 0.1007. In some embodiments, methods as disclosed herein B₂ is about 0.045-0.55. In some embodiments, methods as disclosed herein, B₂ is 0.0493.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1. Image illustrating 30 degree portions in a clock hour distribution relative to the temporal (T) and nasal (N) retinal positions

FIG. 2A-2C. Optical coherence tomography (OCT) from 3 patients with elevation of bilateral optic nerves on examination. FIG. 2A. A patient with mild disc edema due to idiopathic intracranial hypertension. FIG. 2B. The baseline OCT of a patient with anomalous nerves. OCT measurements stable over 8 months and unchanged with trial of acetazolamide. FIG. 2C. A patient with pseudopapilledema from buried optic nerve drusen confirmed with ultrasonography. This patient had been referred by a community provider to the emergency department for urgent papilledema evaluation. The clock-hour measurements are elevated in one localized area in FIG. 2B and FIG. 2C, whereas multiple clock hours in the papilledema patient are elevated. This is the basis of the hypothesis that more clock hour variability would be seen in papilledema patients than pseudopapilledema patients. BNFL, retinal nerve fiber layer.

FIG. 3. Mean retinal nerve fiber layer (RNFL) thickness for the papilledema and pseudopapilledema groups. The papilledema group has a larger RNFL, although there is significant overlap between the 2 groups. OCT, optical coherence tomography.

FIG. 4. Absolute consecutive differences for the papilledema and pseudopapilledema groups. The papilledema group showed more variability, although there is overlap between the 2 groups.

FIG. 5. Optic Disc Edema Index. Linear combination formula for covariates 1) the mean retinal nerve fiber layer (RNFL) thickness and 2) the mean absolute consecutive difference. ^{β}1=0.1007, ^{β}2=0.0493, O̅C̅T̅=average RNFL thickness, A̅b̅s̅D̅i̅f̅f̅=mean absolute consecutive difference.

FIG. 6. Area under the curve (AUC) for the receiver operating characteristics (ROC) curve using the worst eye to calculate a linear combination. Covariates were 1) the mean retinal nerve fiber layer thickness and 2) the mean absolute consecutive difference.

FIG. 7. Calibration curve plot for the linear combination model. This demonstrates good calibration for the linear model. The mean absolute error=0.015.

FIG. 8. Frequency plot of the outcome values, or Optic Disc Edema Indices, for the linear combination formula applied to this data set. There is little overlap between the 2 groups.

FIG. 9. Mean retinal nerve fiber layer (RNFL) thickness for the papilledema and pseudopapilledema groups. OCT, optical coherence tomography.

FIG. 10. Absolute consecutive differences for the papilledema and pseudopapilledema groups.

FIG. 11. Area under the curve (AUC) for the receiver operating characteristics (ROC) curve using the worst eye to calculate a linear combination.

FIG. 12. Frequency plot of the outcome values, or Optic Disc Edema Indices, for the linear combination formula applied to this data set.

FIG. 13. Area under the curve (AUC) for the receiver operating characteristics (ROC) curve using the worst eye to calculate a linear combination.

FIG. 14. Frequency plot of the outcome values, or Optic Disc Edema Indices, for the linear combination formula applied to this data set.

FIG. 15. Mean retinal nerve fiber layer (RNFL) thickness for the papilledema and pseudopapilledema groups. OCT, optical coherence tomography.

FIG. 16. OCT magnitude as a function of age for the papilledema and pseudopapilledema groups.

FIG. 17. Frequency plot of the outcome values, or Optic Disc Edema Indices, for the linear combination formula applied to this data set.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

Differentiating patients with papilledema from those with pseudopapilledema has been a long-standing dilemma for not only the comprehensive ophthalmologist but also for well-established neuro-ophthalmologists (1). It is important to be able to correctly diagnose and distinguish these two conditions because failure to recognize true papilledema can have serious life-threatening and vision-threatening complications (2), while misclassifying pseudopapilledema can lead to multiple unnecessary and invasive tests (1).

There has been much research into diagnostic tools to aid the clinical examination in this regard (3-6). Previous studies have evaluated ultrasonography (7,8), optical coherence tomography (OCT) (8-16), OCT angiography (17), and fluorescein angiography (18). Although all these tests have utility, each has advantages and disadvantages in the clinical setting. OCT is user friendly, accessible in most ophthalmology practices, and can be used for both young and elderly patients.

The presently-disclosed subject matter includes a method for differentiating between papilledema and pseudopapilledema. Methods as disclosed herein involve determining retinal nerve fiber layer (RNFL) thickness of an elevated optic nerve in a subject. Magnitude and variability of the RNFL thickness are considered.

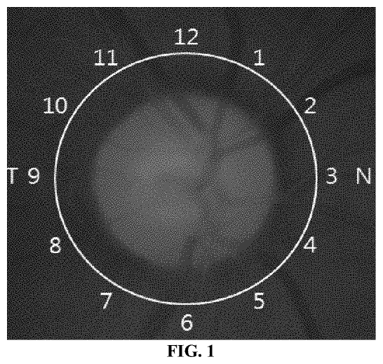

In some embodiments of the presently-disclosed subject matter the RNFL thickness of an elevated optic nerve of a subject is determined for each of twelve clock-hour positions, as illustrated in FIG. 1. In particular, FIG. 1 illustrates 30 degree portions in a clock-hour distribution relative to the temporal (T) and nasal (N) retinal positions.

The RNFL thickness for the n clock hour position can be referred to as Yₙ. As such, the RNFL thickness at the hour 1 position is Y₁, the RNFL thickness at the hour 2 position is Y₂, and so forth.

In some embodiments, methods as disclosed herein involve calculating a magnitude value (M), where M=(Y₁+Y₂+Y₃+Y₄+Y₅+Y₆+Y₇+Y₈+Y₉+Y₁₀+Y₁₁+Y₁₂)/12.

In some embodiments, methods as disclosed herein involve calculating a variability value (V), where V=(Y₁−Y₁₂)+(Y₂−Y₁)+(Y₃−Y₂)+(Y₄−Y₃)+(Y₅−Y₄)+(Y₆−Y₅)+(Y₇−Y₆)+(Y₈−Y₇)+(Y₉−Y₈)+(Y₁₀−Y₉)+(Y₁₁−Y₁₀)+(Y₁₂−Y₁₁).

In some embodiments, methods as disclosed herein involve calculating a magnitude-variability value (X), where X=(B₁*M)+(B₂*V), B₁ is a magnitude coefficient, and B₂ is a variability coefficient.

In some embodiments, methods as disclosed herein B₁ is about 0.08-0.12. In some embodiments, methods as disclosed herein B₁ is 0.1007. In some embodiments, methods as disclosed herein B₂ is about 0.045-0.55. In some embodiments, methods as disclosed herein, B₂ is 0.0493.

In some embodiments, methods as disclosed herein involve detecting the RNFL thickness for each of twelve clock-hour positions using optical coherence tomography (OCT).

Some embodiments of the presently-disclosed subject matter involve a method for differentiating between papilledema and pseudopapilledema, which involves identifying a subject with an elevated optic nerve, detecting in the subject a retinal nerve fiber layer (RNFL) thickness at each of twelve clock-hour positions using optical coherence tomography (OCT).

In some embodiments, the method includes identifying the subject as having pseudopapilledema when X is less than about 13.2. In some embodiments, the method includes identifying the subject as having papilledema when Xis greater than about 13.2. In some embodiments, the method includes identifying the subject as having papilledema when X is greater than about 14.8. X can be calculated using the following formula:

X=(B₁*M)+(B₂*V)

In some embodiments, the method involves administering treatment for papilledema when X is greater than 13.2. In some embodiments, the method involves administering treatment for papilledema when X is greater than 14.8.

The presently-disclosed subject matter includes a method for determining a magnitude and variability of retinal nerve fiber layer (RNFL) thickness of an elevated optic nerve in a subject, which involves detecting in the subject a retinal nerve fiber layer (RNFL) thickness for each of twelve clock-hour positions, calculating a magnitude value (M), calculating a variability value (V), and calculating a magnitude-variability value (X).

The presently-disclosed subject matter includes a method for differentiating between papilledema and pseudopapilledema, which involves identifying a subject with an elevated optic nerve; detecting in the subject a retinal nerve fiber layer (RNFL) thickness at each of twelve clock-hour positions using optical coherence tomography (OCT); and identifying the subject as having pseudopapilledema when X is less than 13.2, and declining to identify the subject has having pseudopapilledema when X is greater than 13.2.

In some embodiments of the method for differentiating between papilledema and pseudopapilledema, the method also includes identifying the subject as having papilledema when X is greater than 13.2. In some embodiments, the method further includes administering treatment for papilledema when X is greater than 13.2.

In some embodiments of the method for differentiating between papilledema and pseudopapilledema, the method also includes identifying the subject as having papilledema when X is greater than 14.8. In some embodiments, the method further includes administering treatment for papilledema when X is greater than 14.8.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

Although any methods, devices, and materials similar or equivalent to those described herein can be used in the practice or testing of the presently-disclosed subject matter, representative methods, devices, and materials are described herein.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±0.5%, in some embodiments ±0.1%, in some embodiments ±0.01%, in some embodiments ±0.001% from the specified amount, and in some embodiments ±0.0001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The presently-disclosed subject matter is further illustrated by the following specific but non-limiting examples. The following examples may include compilations of data that are representative of data gathered at various times during the course of development and experimentation related to the present invention.

### EXAMPLES

### Example 1

A retrospective review was conducted of patients who presented to a single fellowship-trained neuro-ophthalmologist (RL) with clinically elevated optic nerves over a 4-year period. Patients were included if they had a diagnosis of papilledema or pseudopapilledema, which included optic disc drusen. Both eyes of every patient were included for analysis.

The diagnosis of papilledema required a documented lumbar puncture with opening pressure greater than 24 cm H2O and appropriate neuroimaging. Further inclusion criteria were age 18 years and reliable Cirrus OCT optic nerve head measurements (Carl Zeiss Meditec, Inc, Dublin, Calif.). Both eyes had to have some degree of disc edema but they could be asymmetric. Exclusion criteria included Grade 5 papilledema due to presumably unreliable OCT scans, intracranial or ocular pathology (e.g., masses, ischemic lesions, and panretinal photocoagulation) that may have a confounding effect on the RNFL because of anterograde or retrograde axonal degeneration (19), and other co-existing optic nerve pathology.

Inclusion criteria for pseudopapilledema were patients with clinically elevated optic nerves who had been referred to neuro-ophthalmology. Any questionable diagnoses from the neuro-ophthalmologist were excluded. Etiologies for pseudopapilledema included optic disc drusen and congenital anomaly; other etiologies were excluded. A lumbar puncture was not required as the standard of practice at the institution does not routinely order lumber punctures for these patients.

Data collected included age of patient, diagnosis, and lumbar puncture opening pressure if performed. Collected OCT data included the mean RNFL thickness, the 4 quadrant RNFL thickness measurements, and the 12 clock-hour RNFL thickness measurements that are all readily available in the standard OCT report. The clock hours on the right eye were labeled clockwise and the left eye was labeled counterclockwise to maintain consistency with temporal and nasal sides.

The mean of the 12 clock-hour RNFL thickness and the mean absolute consecutive difference for clock hours 1-12_{(MACD1-12)} were compared between papilledema and pseudopapilledema groups using a mixed-effect model adjusting for age and clock hour with a random intercept for subjects and eyes (nested within subject). This accounted for within-patient and inter-eye correlations. Sex was not controlled for because it has not been showed to affect the RNFL (20). MACD₁₋₁₂ was defined as AbsDiff=(|OCThour1−OCThour12|+|OCThour2−OCThour1|+ . . . +|OCThour12−OCThour11|)/12. A linear risk score of RNFL and MACD1-12 was developed to distinguish papilledema from pseudopapilledema with their coefficients derived from a logistic regression model. The area under the curve (AUC) for the receiver operating characteristics (ROC) curve and calibration curve was used to evaluate potential clinical usage.

The calibration curve was developed using R 3.6.1 with the rms package (Regression Modeling Strategies. R package version 5.1-3.1. CRAN.R-project. org/package=rms). The ROC was plotted for sensitivity against 1-specificity. The worse eye (larger linear combination) was used to calculate the ROC. The AUC confidence interval (CI) was calculated using a bootstrap method (2000 replicates). Four criteria were used to find the optimal cutoff (Youden (21), Closest to the top left (22), Index of Union (22), and the Concordance Probability Method (23)). A 2-sided P value less than 0.05 was considered statistically significant.

The development of clock-hour data from OCT measurements has allowed increased sensitivity to detect conditions that might have asymmetric involvement of the optic nerve head. Based on clinical observation, patients with pseudopapilledema either from drusen or a congenital anomaly often have only part of the nerve that is elevated. This is in contrast to patients with papilledema who have clock-hour elevation that varies relatively linearly based on the magnitude of the normal nerve fiber layer thickness.

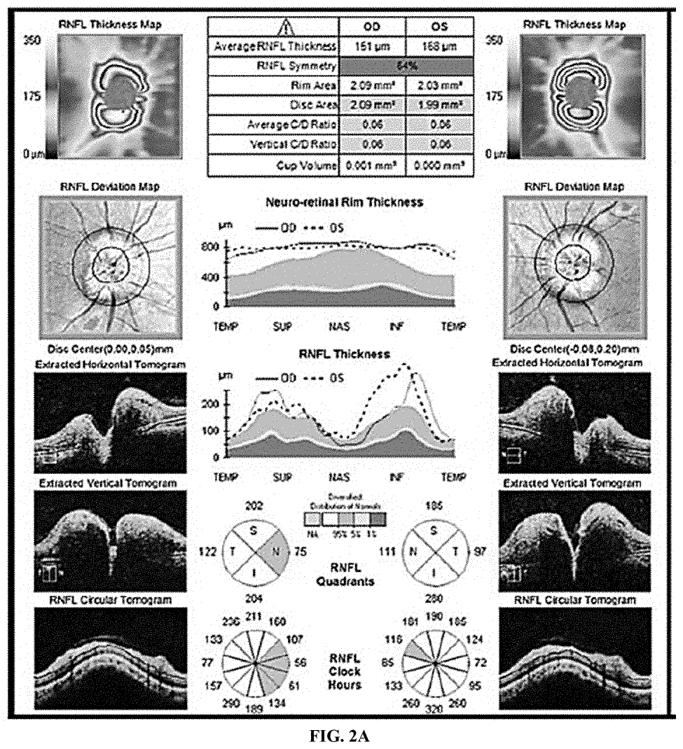

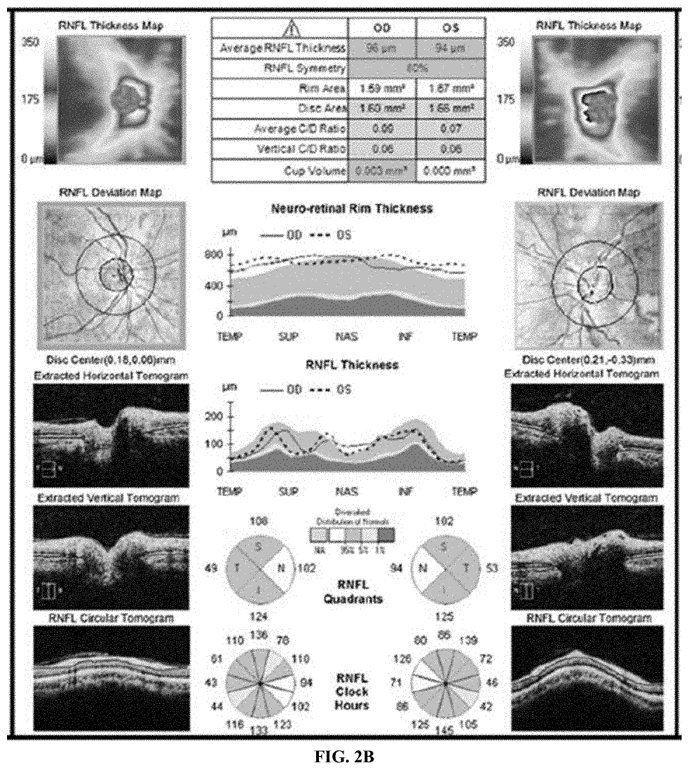

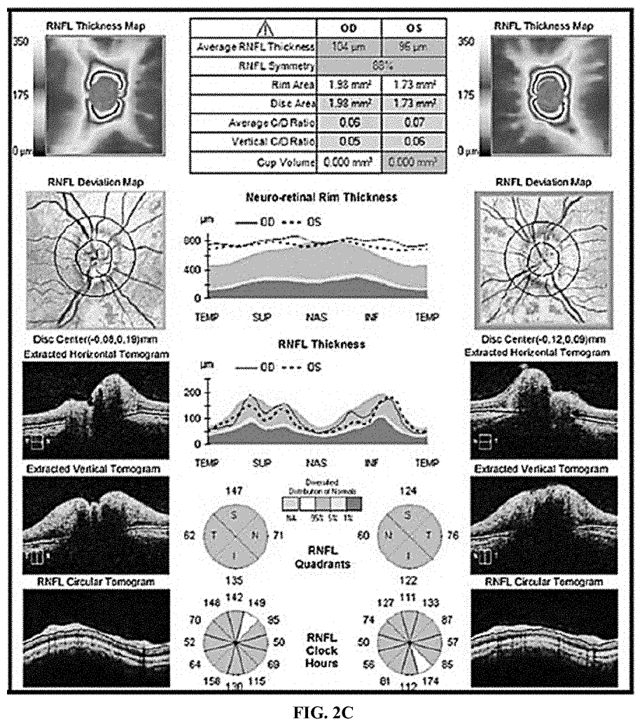

FIGS. 2A-2C illustrates this observation with an example of an OCT scan from patients having mild disc edema, optic nerve drusen, and congenitally anomalous nerves. All 3 patients had been referred with a concern for papilledema from community eye doctors.

Based on the above observation, it was contemplated that patients with papilledema would have more variability in their individual clock-hour measurements than those with pseudopapilledema and that this variability in the OCT clock-hour data could be used to distinguish patients with papilledema from those with pseudopapilledema. A linear risk score was devised using those data to separate the 2 groups, and this model is presented herein.

One hundred sixteen eyes (58 patients) were found that met the criteria for analysis (Table 1). Forty-four eyes (22 patients) had papilledema, and 72 eyes had pseudopapilledema; of the 72 eyes, 36 eyes had optic disc drusen and 36 had anomalous nerves. Of the eyes that had optic nerve drusen, 20 eyes were visible on examination. The rest were buried and diagnosed by ultrasonography or seen on OCT.

TABLE 1
Demographic information of the papilledema
and pseudopapilledema groups.

| | Papilledema | | Pseudopapilledema | |
|---|---|---|---|---|
| Total number (eyes) | 22 | (44) | 36 | (72) |
| Age, yr (range, SD) | 28.3 | (19-49, 7.5) | 40.6 | (18-89, 19.7) |

| | | |
|---|---|---|
| Gender (female/total) | 22/22 | 27/36 |
| Diagnosis | 21/22 IIH | 18/36 drusen (10 |
| | | visible drusen) |
| Frisen grade | Frisen Grade 1: 26 | N/A |
| | Frisen Grade 2: 13 | |
| | Frisen Grade 3: 3 | |
| | Frisen Grade 4: 1 | |
| Lumbar puncture, | | N/A |
| cm H2O (range, SD) | | |

The average age of the papilledema group was 28.3 years (range=19-49 years, SD=7.5 years), and all were women. Twenty-one of the 22 patients were diagnosed with idiopathic intracranial hypertension (IIH). The remaining patient had hydrocephalus from a ventricular plexus choroid papilloma. Of the 44 eyes, 26 eyes had Frisen Grade 1 papilledema, 13 eyes were Grade 2, 3 were Grade 3, and 1 was Grade 4. Twenty-one of the 22 patients had a documented opening pressure by lumbar puncture. The mean opening pressure was 36.8 cm H2O, (range=25.7-55 cm H2O, SD 8.0 cm H2O). The one patient with hydrocephalus had an external ventricular drain placed with an intra-cranial pressure of 25 cm H2O.

The average age of the patients with pseudopapilledema without drusen was 40.6 years (range=18-89 years, SD=19.7 years), and the average age of the patients with pseudopapilledema with optic nerve drusen was 53.9 years (range=29-80 years, SD=12.7 years). Twenty-seven of the 36 patients with pseudopapilledema were women.

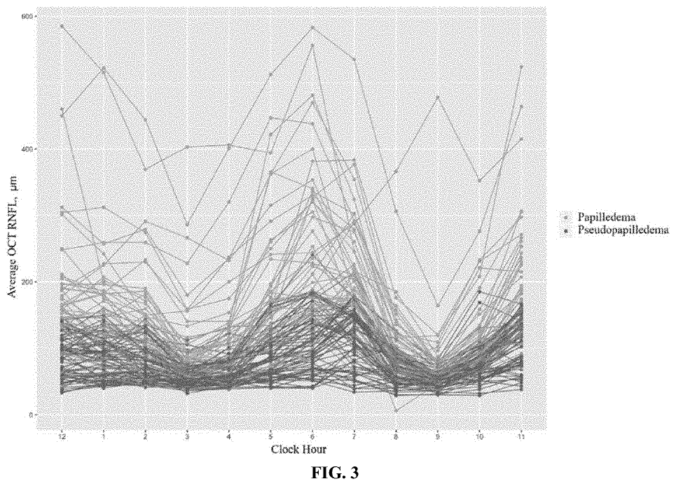

The average RNFL thickness for the papilledema group was higher than that for the pseudopapilledema group (papilledema=163±68 mm, pseudopapilledema=82±22 mm, P<0.001). As can be seen in FIG. 3, the papilledema group overall had thicker RNFLs; however, there was notable overlap between the 2 groups.

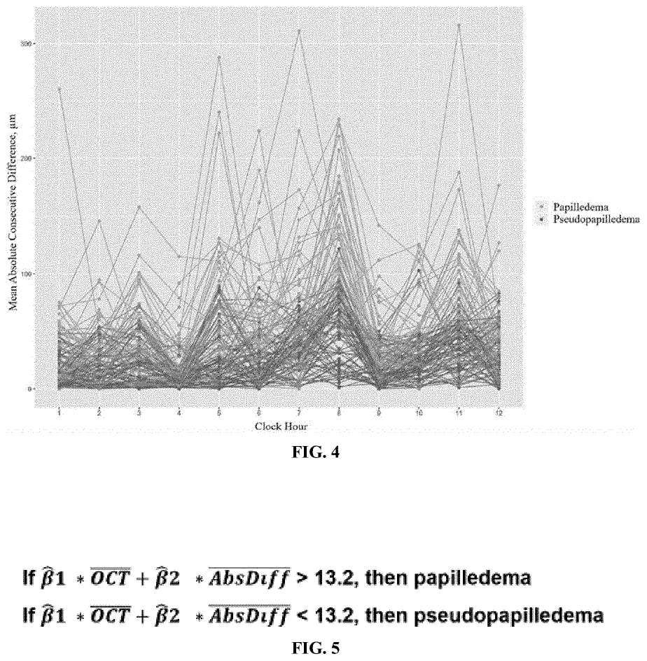

The papilledema group also had higher MACD1-12 (papilledema=57±20 mm, pseudopapilledema=26±11 mm, P<0.001). The papilledema group had more variability from 1 clock hour to the next, but there was still overlap between the 2 groups (FIG. 4).

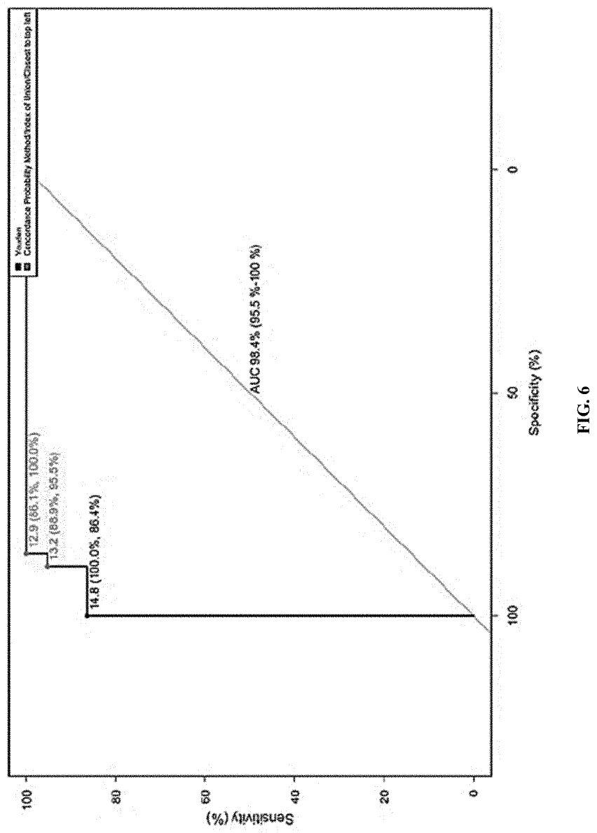

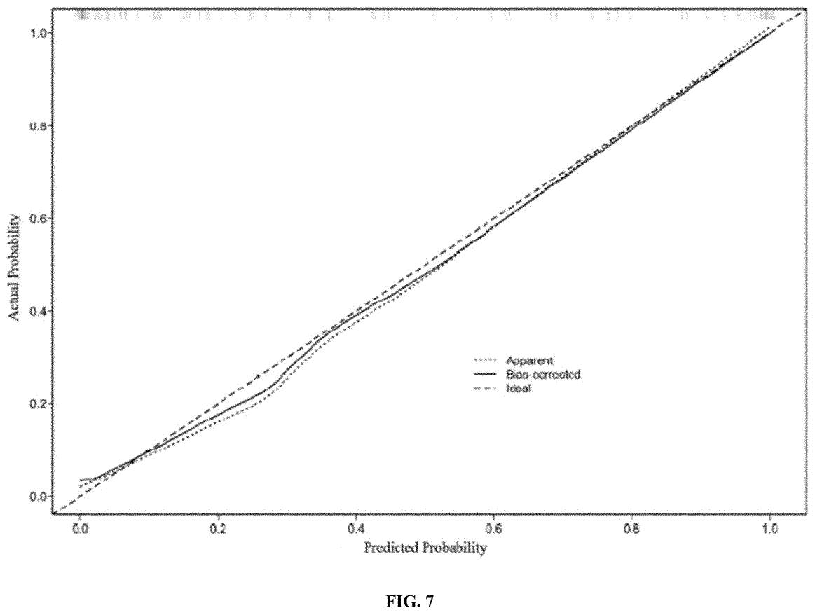

From these data, a linear risk score was created with an outcome metric (termed the Optic Disc Edema Index [ODEI]) to better stratify papilledema nerves from pseudo-papilledema (FIG. 5). When the linear combination of the average values for covariates 1) the RNFL thickness and 2) the mean absolute consecutive difference were used to classify the 2 groups, the AUC of 98.4% (95% CI 95.5%-100%) was achieved (FIG. 6). As can be referenced in FIG. 7, good calibration was obtained for this linear combination (the mean absolute error=0.015). With different cut-off values as shown in FIG. 6, sensitivity ranged from 86% to 100% and specificity from 86% to 100%.

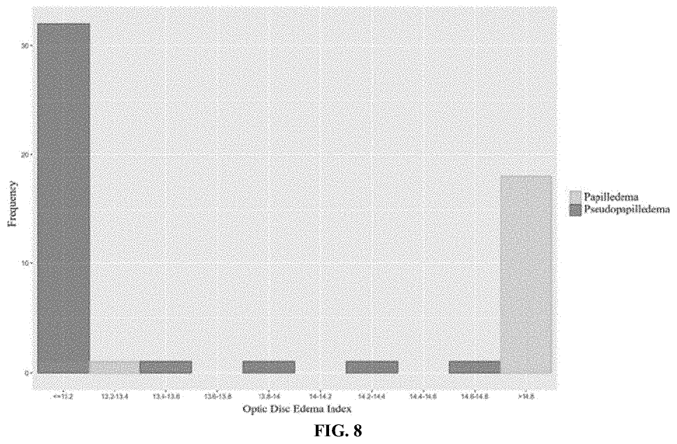

Table 2 shows the distribution of the linear combination results for this data set. This was developed using the worst eye from each patient. Nearly all the papilledema eyes had a value of greater than 14.8; 4 eyes were less than 14.8. Nearly all the pseudopapilledema eyes had a linear combination value of less than 13.2; 4 eyes were greater than 13.2. FIG. 8 graphically shows how there is a separation between the 2 groups with only a small area of overlap.

TABLE 2
Frequency distribution of the linear combination formula
values for papilledema and pseudopapilledema groups.

| Linear Combination | Papilledema | Pseudopapilledema |
|---|---|---|
| Value | N = 22 | N = 36 |
| <13.2 | 0.05 (1)* | 0.89 (32) |
| 13.2-13.4 | 0.05 (1) | 0.00 (0) |
| 13.4-13.6 | 0.05 (1) | 0.03 (1) |
| 13.6-13.8 | 0.00 (0) | 0.00 (0) |
| 13.8-14.0 | 0.00 (0) | 0.03 (1) |
| 14.0-14.2 | 0.00 (0) | 0.00 (0) |
| 14.2-14.4 | 0.00 (0) | 0.03 (1) |
| 14.4-14.6 | 0.00 (0) | 0.00 (0) |
| 14.6-14.8 | 0.05 (1) | 0.03 (1) |
| >14.8 | 0.82 (18) | 0.00 (0) |

The worst eye was used from each patient to calculate a risk score. Most papilledema eyes had values greater than 14.8, whereas most pseudopapilledema eyes had values less than 13.2.
*Numbers after proportions are frequencies.

The opening pressures on lumbar puncture for the 4 patients with papilledema with an ODEI, 14.8 were 27, 33, 34, and 39 cm H₂O. Three of the 4 were described to have mild disc elevation only in the nasal quadrant. The fourth had Frisen Grade 2 disc edema in one eye and Grade 1 in the second eye. All 4 patients had been started on acetazolamide by outside providers before presentation. Four patients with pseudopapilledema had an ODEI of greater than 13.2. One of these 4 had a lumbar puncture with normal opening pressure. Two patients had stable pseudopapilledema over a 2-year period. One patient was a 49-year-old obese woman who had sleep apnea and intermittently experienced pulsatile tinnitus but no headache. Clinical examination and OCT were noted consistent with pseudopapilledema with no true papilledema. The examination and OCT were stable over a 2-month period.

The importance of recognizing papilledema cannot be overestimated because it typically indicates a vision-threatening and often life-threatening problem. Accordingly, recognizing pseudopapilledema and being able to distinguish it adequately from true papilledema are similarly important. Unfortunately, it often remains difficult to determine true vs pseudopapilledema (1).

Previous investigators have used measurements of RNFL thickness from OCT readings to separate true from pseudopapilledema. Carta et al (10) compared the 4 quadrant measurements and found that RNFL thickness was greater in papilledema, particularly the inferior quadrant. Another study observed that the average RNFL thickness was greater in papilledema for all 12 clock-hour segments than that in pseudopapilledema in younger patients (11). Bassi and Mohana also found that papilledema nerves had a thicker RNFL than pseudopapilledema, but the highest AUC that they achieved for stratification was 0.79 using the nasal quadrant (14). Lee confirmed these findings with an AUC of the nasal quadrant of 0.86 (15). The results agree with these observations, but a higher AUC of 0.98 is reported using the linear model described herein. An overlap of the RNFL thickness was observed with the 2 groups and believed this metric was limited in its ability to reliably and accurately separate them. Kulkarni et al did not find a difference in RNFL thickness between papilledema eyes and buried optic nerve drusen (16) further supporting the notion that RNFL thickness alone is not a sufficient biomarker to separate these groups.

In 2018, Costello et al (1) published a review of different OCT modalities used to compare papilledema and pseudopapilledema, including the subretinal hyporeflective space (9,12) and the angle of the Bruch membrane. The study by Johnson et al compared optic disc edema with optic nerve head drusen. They again found that the disc edema nerves had thicker RNFLs than the drusen nerves, agreeing with the above. The ROC for RNFL thickness was able to differentiate the 2 with sensitivity ranging from 65% to 80% and specificity from 70% to 80%. This was limited by the overlap of the 2 groups, similar to the results. The ROC for the subretinal hyporeflective space was able to give a sensitivity of 75% and specificity of 90%. The linear model had higher sensitivity and specificity. Some have analyzed the angle of the Bruch membrane in papilledema nerves before and after lumbar puncture (24,25) and in comparison to disc edema from other etiologies (26). However, as Costello et al (1) point out, this has not been reproducible in every study (16) and has been seen in normal optic nerves (27).

Carter et al reported a large cohort of 407 patients who underwent ultrasonography (7). They reported a sensitivity of 90% and specificity of 79% for the 30° test. Although these results seem promising, it does require specialized staff to perform the test.

Chang et al reported a high accuracy rate of 97% for fluorescein angiography to detect drusen vs optic disc edema (6). However, this is an invasive test requiring an intravenous line, skilled staff, is more time intensive than OCT, has medication related side effects, and is contraindicated in pregnancy. These negatives are contrasted with the relatively few negatives of the OCT.

The clinical observation that the variability in the clock-hour data seemed to differ between the patients with papilledema and pseudopapilledema gave rise to the hypothesis tested in this article. Analysis found that patients with papilledema have an increase in the mean

RNFL thickness than patients with pseudopapilledema. However, these distributions overlap; thus, the 2 groups could not be separated exclusively using this variable with high enough sensitivity and specificity. It was also found that an increase in absolute consecutive clock-hour variability was seen in both pseudo and true papilledema; these distributions also had a significant overlap and were not sufficient to separate the 2 groups.

Therefore, a linear model was devised to best fit the variables of 1) the overall thickness of the retinal nerve fiber layer and 2) the absolute consecutive difference between clock-hour segments. The outcome metric, which is called the ODEI, distinguished between the 2 conditions with high sensitivity and specificity. The data show that an ODEI>14.8 was associated with papilledema, whereas, 13.2 was associated with pseudopapilledema. Accordingly, one can use this outcome metric to determine the likelihood of optic disc edema. The AUC for this model was 0.98, which indicates an excellent fit of this linear model. In the series of 116 eyes, only 4 papilledema eyes had optic nerve edema indexes less than 14.8 and 4 pseudopapilledema eyes greater than 13.2. Thus, there remains a small but important area of overlap using this metric.

Conveniently, the clock-hour data are readily available with current software, and the OCT machine is widely accessible, making clinical implementation of this method practical. This makes the linear model attractive to not only neuro-ophthalmologists but also referring comprehensive ophthalmologists to better triage and manage these patients.

### Example 2

A formula based on the retinal nerve fiber layer (RNFL) thickness and clock hour variability was created, as described above, which separated the two diagnoses with an AUC of 98.4%. In the present Example, this formula was validated on an independent cohort of patients.

Institutional prospective IRB approval was obtained through Vanderbilt University Medical Center, and the Declaration of Helsinki was adhered to. A retrospective review was conducted of patients who presented to a single fellowship trained neuro-ophthalmologist (RL) with clinically elevated optic nerves over a four-year period (2016-2020). Patients older than 18 years of age were included if they had a diagnosis of papilledema or pseudopapilledema, which included optic disc drusen, and reliable Cirrus OCT optic nerve head measurements (Carl Zeiss Meditec, Inc, Dublin, Calif.). Both eyes of every patient were included for analysis. Similar inclusion and exclusion criteria and statistical analysis were applied to this cohort as the original study with the notable exception in that patients with stable pseudopapilledema with a history of papilledema were included in the pseudopapilledema group. These are summarized below.

Papilledema patients were included if they were diagnosed with Idiopathic Intracranial Hypertension (IIH) based on the modified Dandy Walker Criteria from the IIH Treatment Trial (IIHTT) or had a documented lumbar puncture with opening pressure greater than 24 cm H2O and appropriate neuroimaging. Both eyes had to have some degree of disc edema, but they could be asymmetric. Exclusion criteria included grade 5 papilledema due to presumably unreliable OCT scans, intracranial or ocular pathology (e.g. masses, ischemic lesions, panretinal photocoagulation) which may have a confounding effect on the RNFL due to anterograde or retrograde axonal degeneration (19), or other co-existing optic nerve pathology like myelinated nerve fiber layer.

Inclusion criteria for pseudopapilledema were patients with clinically elevated optic nerves who had been referred to Neuro-ophthalmology. Any questionable diagnoses from the Neuro-ophthalmologist were excluded. Etiologies for pseudopapilledema included optic disc drusen and congenital anomaly. Different for this cohort compared to the previous study is that patients could have stable pseudopapilledema after having a history of papilledema. Stability was defined as no clinical concerns for active papilledema in addition to a stable exam and diagnostic tests over a period of 3 months. Other etiologies were excluded. A lumbar puncture was not required as the standard of practice at the institution does not routinely order lumber punctures for patients with pseudopapilledema.

Data collected included age of patient, diagnosis, and lumbar puncture opening pressure if performed. Collected OCT data included mean RNFL thickness, the four quadrant RNFL thickness measurements, and the twelve clock-hour RNFL thickness measurements which are all readily available in the standard OCT report. The clock hours on the right eye were labeled clockwise, and the left eye was labeled counterclockwise in order to maintain consistency with temporal and nasal sides. The OCT variables analyzed were the mean of the twelve clock-hour RNFL thickness and its mean absolute consecutive difference for clock hours 1-12 (MACD₁₋₁₂) defined as: A̅b̅s̅D̅i̅f̅f̅=(|OCThour1−OCThour12|+|OCThour2−OCThour1|+ . . . +|OCThour12−OCThour11|)/12 These variables were compared between papilledema and pseudopapilledema groups using mixed effect model adjusting for age and clock hour with random intercept for subjects and eyes (nested within subject). Gender was not controlled for as it has not been showed to affect RNFL. (20) Two-sided p-value less than 0.05 was considered statistically significant.

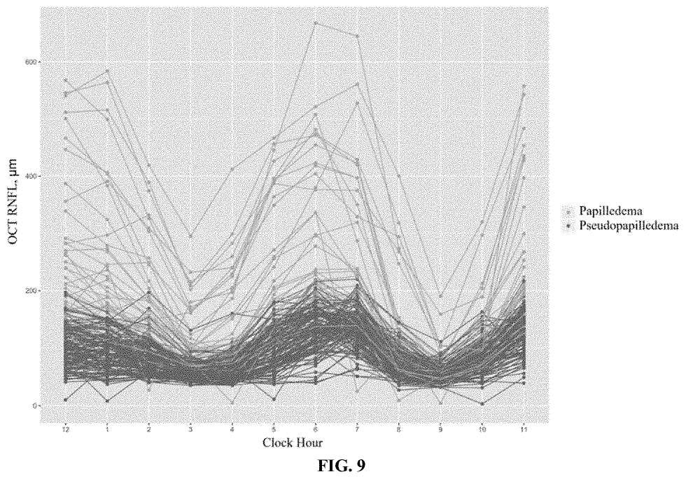

The area under the curve (AUC) for the receiver operating characteristics (ROC) curve was plotted in order to validate the Optic Disc Edema Index (FIG. 9) with this independent cohort. The ROC was plotted for sensitivity against 1-specificity. The worse eye (larger linear combination) was used to calculate the ROC. The AUC confidence interval (CI) was calculated using bootstrap method (2000 replicates). The distribution of the Optic Disc Edema Indices for each patient was also evaluated. The ROC curve was then plotted using the combined data from the original study and this study.

One hundred and seventy-six eyes (88 patients) were found that met the criteria for analysis (Table 3). Forty-six eyes (23 patients) had papilledema and 130 eyes (65 patients) had pseudopapilledema. Of the pseudopapilledema group, 16 eyes had optic disc drusen, 46 eyes had anomalous nerves, and 11 had stable pseudopapilledema after a history of papilledema. Of the eyes that had optic nerve drusen, 14 were visible on exam. One patient had buried drusen that was confirmed with ultrasonography.

TABLE 3
Frequency distribution of the linear combination formula
values for papilledema and pseudopapilledema groups.

| Linear Combination | Papilledema | Pseudopapilledema |
|---|---|---|
| Value | N = 23 | N = 65 |
| <=13.2 | 0.09 (2)^{a} | 0.89 (58) |
| 13.2-13.4 | 0.00 (0) | 0.03 (2) |
| 13.4-13.6 | 0.04 (1) | 0.00 (0) |
| 13.6-13.8 | 0.00 (0) | 0.00 (0) |
| 13.8-14.0 | 0.00 (0) | 0.02 (1) |
| 14.0-14.2 | 0.00 (0) | 0.02 (1) |
| 14.2-14.4 | 0.00 (0) | 0.00 (0) |
| 14.4-14.6 | 0.00 (0) | 0.03 (2) |
| 14.6-14.8 | 0.00 (0) | 0.00 (0) |
| >14.8 | 0.87 (20) | 0.02 (1) |

The worst eye was used from each patient to calculate a risk score. Most papilledema eyes had values greater than 14.8, while most pseudopapilledema eyes had values less than 13.2.
^{a}Numbers after proportions are frequencies.

The average age of the papilledema group was 28.5 years (range=18-56 years, standard deviation=9.2 years) and 21/23 were female. Twenty patients were diagnosed with idiopathic intracranial hypertension (IIH); three patients had increased intracranial pressure from venous sinus thrombosis. Of the 46 eyes, 31 eyes had Frisen grade 1 papilledema; 6 eyes were grade 2; 11 were grade 3; none were grade 4. The mean opening pressure was 33 cm H2O, (range=21-46 cm H2O, standard deviation 7.0 cm H2O). The two patients with lumbar puncture opening pressure less than 25 cm H2O met criteria for IIH based on MRI findings in accordance with the IIHTT.

The average age of the pseudopapilledema patients without drusen was 33.9 years (range=18-66 years, standard deviation=10.1 years), and the average age of the pseudopapilledema patients with optic nerve drusen was 42 years (range=22-61 years, standard deviation=14 years). Fifty-nine of the 65 pseudopapilledema patients were female. Of the 11 pseudopapilledema patients who had a history of papilledema, three patients had 1 visit. They were diagnosed with pseudopapilledema with no concern for concurrent disc edema, and thus were still included. The rest of the patients had confirmed stability on exam for an average of 7.2 months (range 1-18 months).

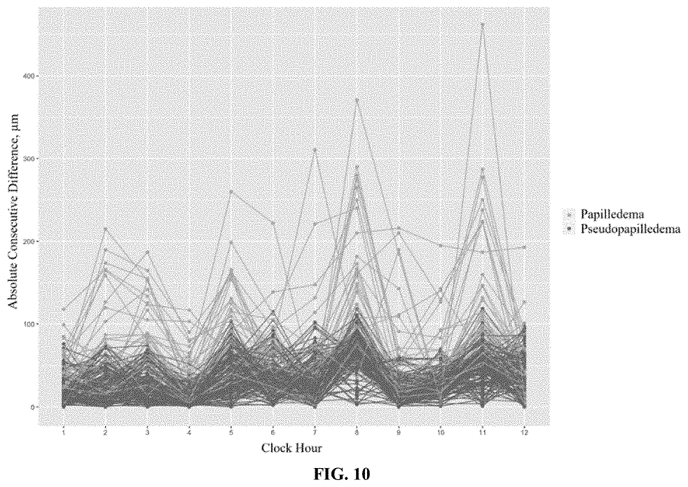

The average RNFL thickness for the papilledema group was higher than that for the pseudopapilledema group (papilledema=178±85 μm, pseudopapilledema=92±16 μm, p<0.001). As can be seen in FIG. 10, the papilledema group overall had thicker RNFLs, however there was notable overlap between the two groups.

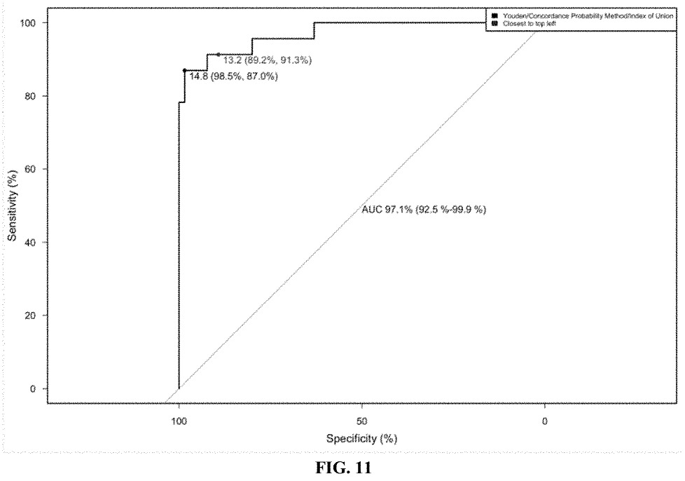

The papilledema group also had higher MACD1-12 (papilledema=66.6±30.8 pseudopapilledema=31.6±6.9 μm, p<0.001). The papilledema group had more variability from one clock hour to the next, but there was still overlap between the two groups (FIG. 11).

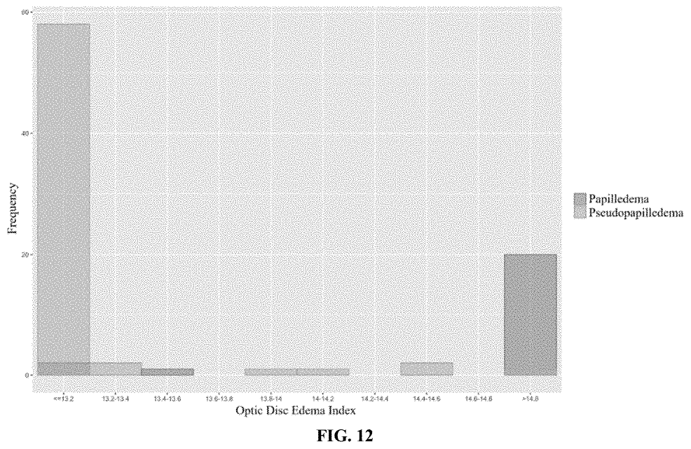

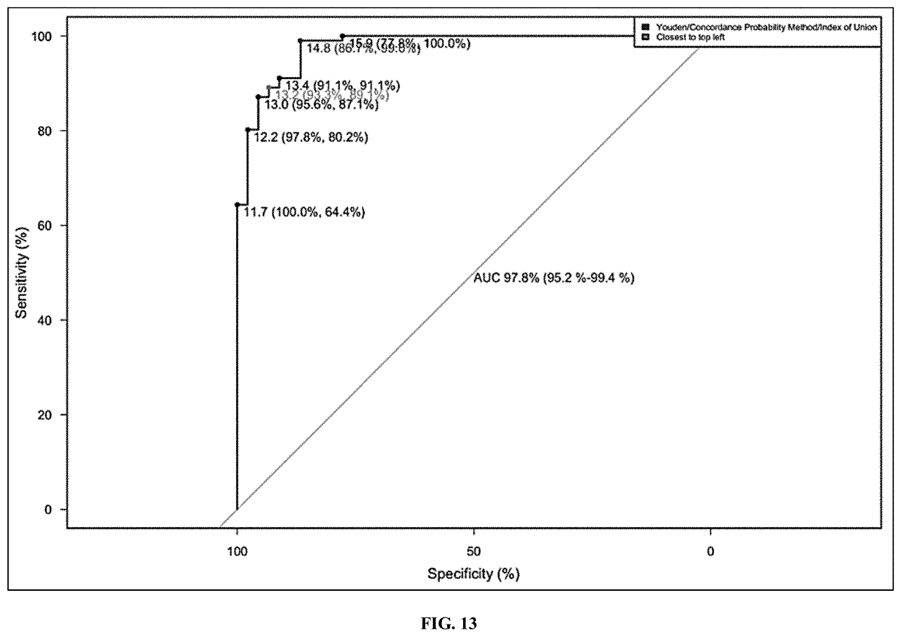

The AUC of the ROC was then plotted to evaluate the ability of the linear combination model, termed Optic Disc Edema Index, to classify the papilledema and pseudopapilledema groups. An AUC of 97.1% (95% CI 92.5-99.9%) was achieved (FIG. 12). A cutoff of 13.2 yielded a sensitivity of 91.3% and specificity of 89.2%. A cutoff of 14.8 yielded a sensitivity of 87% and specificity of 98.5%. When the ODEI for the worst eye of each patient was used, it was found that 20/23 papilledema patients had indices over 14.8 and 58/65 pseudopapilledema patients had indices below 13.2 (FIG. 13, Table 4). Only two papilledema patients had ODEI<13.2 and only one pseudopapilledema patient had ODEI>14.8. Interestingly, the three papilledema patients with ODEI<14.8 had all been started on treatment (lumbar puncture plus acetazolamide) prior to their baseline OCT scan. The seven pseudopapilledema patients with ODEI>13.2 all had above normal RNFL in at least one clock hour.

TABLE 4
Frequency distribution of the linear combination formula values
for the combined papilledema and pseudopapilledema groups
of the original publication and the validation cohorts.

| Linear Combination | Papilledema | Pseudopapilledema |
|---|---|---|
| Value | N = 45 | N = 101 |
| <=13.2 | 0.07 (3)^{a} | 0.89 (90) |
| 13.2-13.4 | 0.02 (1) | 0.02 (2) |
| 13.4-13.6 | 0.04 (2) | 0.01 (1) |
| 13.6-13.8 | 0.00 (0) | 0.00 (0) |
| 13.8-14.0 | 0.00 (0) | 0.02 (2) |
| 14.0-14.2 | 0.00 (0) | 0.01 (1) |
| 14.2-14.4 | 0.00 (0) | 0.01 (1) |
| 14.4-14.6 | 0.00 (0) | 0.02 (2) |
| 14.6-14.8 | 0.02 (1) | 0.01 (1) |
| >14.8 | 0.84 (38) | 0.01 (1) |

The worst eye was used from each patient to calculate a risk score. Most papilledema eyes had values greater than 14.8, while most pseudopapilledema eyes had values less than 13.2.
^{a}Numbers after proportions are frequencies.

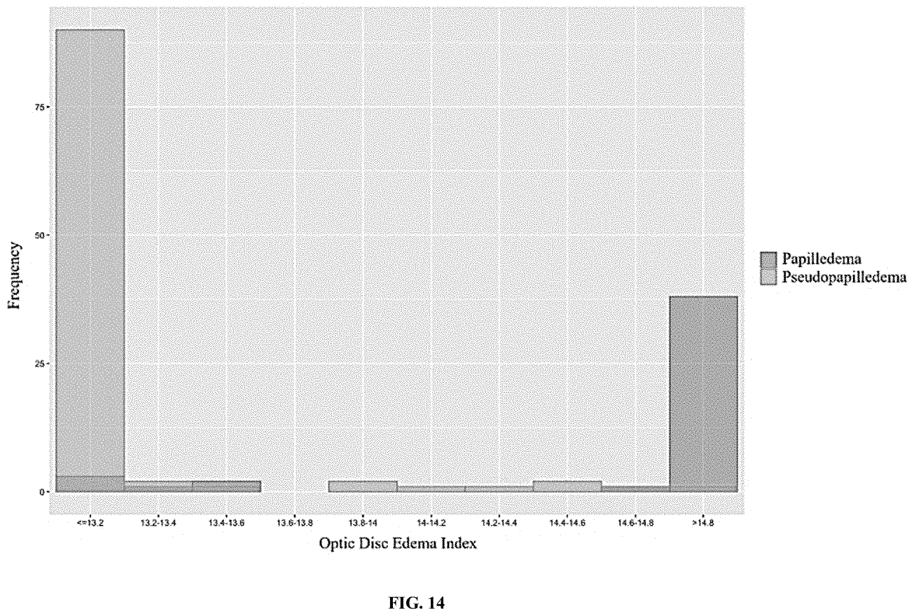

The AUC of the ROC was then plotted for the combined cohorts of the original study and this study for a total of 146 patients (FIG. 13). This yielded an AUC of 97.8% (95% CI 95.2-99.4%). FIG. 14 shows the distribution of ODEI for these 146 patients.

In summary, the previously described Optic Disc Edema Index was validated with an independent cohort of patients. The original findings were reproduced and, again, found a very high AUC of 97.1% supporting the ability of this linear model to differentiate papilledema and pseudopapilledema patients. Previous reports of the AUC for RNFL thickness include 79% (14) and 86% (15). Combining the RNFL thickness and the variability in the clock hour data (MACD₁₋₁₂) allows for an improved way to classifying these groups than just using one of these variables in isolation.

There has been much research into the morphological and anatomic characteristics of both papilledema and pseudopapilledema nerves which is creating a growing body of literature suggesting minute differences between these pathologies that are able to be detected with improved diagnostic capabilities. Costello et al. (1) published a great table and review on different OCT features that could help differentiate optic disc drusen from papilledema due to IIH. These included RNFL thickness, Bruch membrane angle, scleral canal size and others. They do remark that due to variability in RNFL thickness in both of these entities, that the authors did not think this could reliably be used as a way to classify the groups. Though just optic disc drusen and papilledema were not compared, the study does support that this feature can be used when combined with variability. Malmqvist et al (28) studied the relationship between visual field defects and peripapillary RNFL thickness in optic disc drusen, and found mostly nerve fiber layer bundle defects. This supports the idea that optic disc drusen affect only localized areas of the optic nerve head and why the clock hour data is better able to detect a difference. Additionally, there have been several papers evaluating the retinal vessel architecture in optic disc drusen compared to controls and optic disc edema (29) which suggests another anatomical difference. There remains a paucity of literature comparing congenitally anomalous nerves to disc edema.

Thus, there are several biomarkers being evaluated to aid the clinician in making a diagnosis. As these biomarkers become better elucidated it is possible that the combination of different features could lead to even greater ability to classify optic nerves afflicted with different pathologies.

It is suggested that clinicians use the model in conjunction with the patient's history (in regard to symptoms of elevated intracranial pressure), exam and other diagnostic studies in order to aid in the decision to pursue head imaging or invasive studies to evaluate for papilledema. As is with all diagnostic studies, the use of this model can benefit from use with other information and/or models (1). Where this model may be particularly useful for clinicians (e.g optometrists, comprehensive ophthalmologists, neurologists) is in aiding the decision to refer to an outpatient Neuro-ophthalmology clinic or to an Emergency Department. A higher ODEI value would support a more urgent evaluation with neuroimaging (and other appropriate evaluation for elevated intracranial pressure) and a lower value may support more flexibility in outpatient referral.

### Example 3

As described above, the Optic Disc Edema Index (ODEI) is a number derived from a linear model of optical coherence tomography (OCT) measurements that differentiates pseudopapilledema from papilledema in adults. It was shown to have an area under the receiver operating characteristics curve (AUC) of 98.4% with sensitivity of 88.9% and specificity of 95.5%. In the present Example, this formula was validated in the pediatric population.

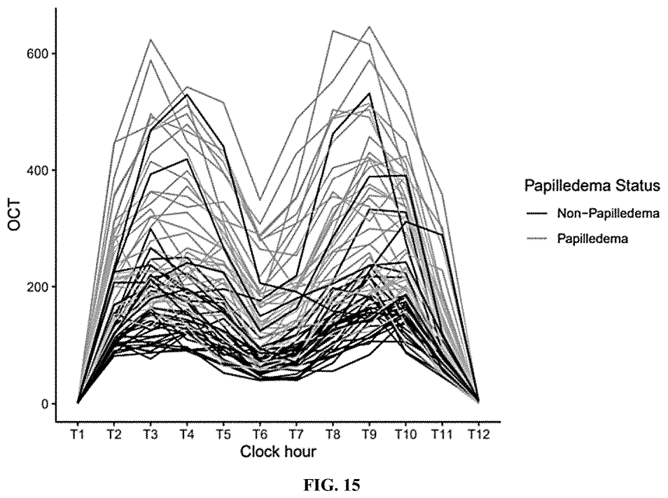

Both eyes of pediatric patients were included for retrospective review and classified as papilledema or pseudopapilledema. The study was verified as IRB exempt. Inclusion, exclusion criteria and analysis were similar to the adult studies. The mean OCT retinal nerve fiber layer thickness and the absolute consecutive difference between adjacent clock hours were compared using mixed-effect models. The AUC and calibration curve were used to evaluate potential clinical usage (FIG. 15).

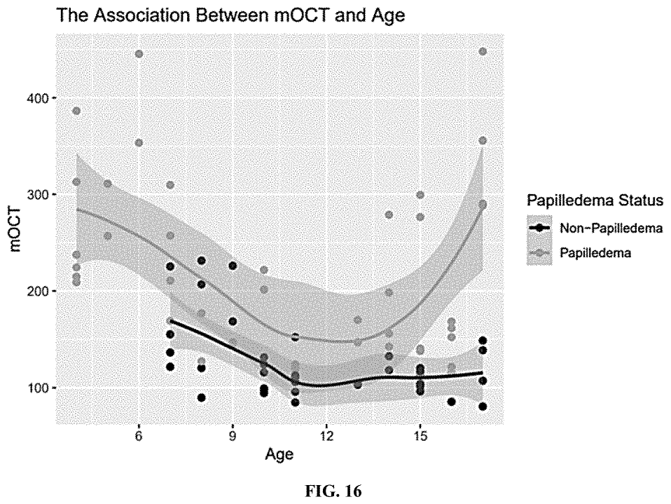

Forty-two (42) eyes with papilledema and 38 with pseudopapilledema were identified. The papilledema group had a higher OCT magnitude (papilledema=222 μm, pseudopapilledema=126 μm, p<0.01) and absolute consecutive difference between clock-hours (papilledema=70.9 μm, pseudopapilledema=45.1 μm, p<0.01). FIG. 16 shows the OCT magnitude as a function of age. Gender was also found to have a significant effect. When the linear combination of these variables was used to classify the groups, AUC of 88.6% (95% CI 80.9-94.6%) was achieved with optimized sensitivity of 78.6% and specificity of 84.2%.

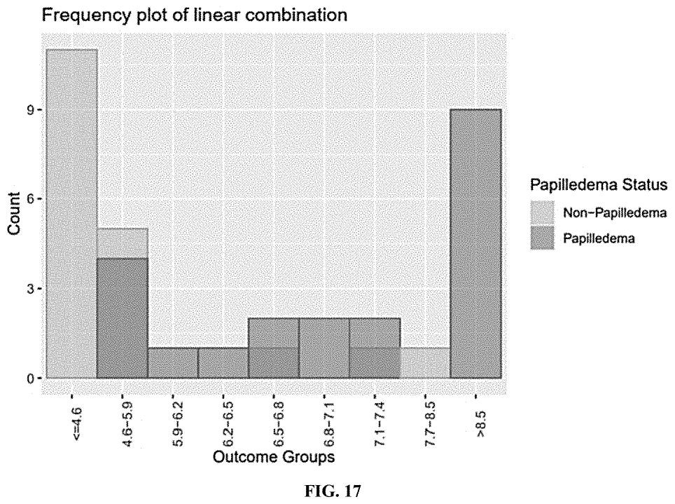

Pediatric patients with papilledema have higher variability and magnitude in OCT measurements than pseudopapilledema. FIG. 17 shows the distribution of ODEI for these patients. The ODEI distinguishes papilledema from pseudopapilledema reliably in the pediatric population, similar to adults.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Costello F, Malmqvist L, Hamann S. The role of optical coherence tomography in differentiating optic disc drusen from optic disc edema. Asia-Pacific J Ophthalmol. 2018; 7:271-279.
2. Rigi M, Almarzouqi S J, Morgan M L, Lee A G. Papilledema: epidemiology, etiology, and clinical management. Eye and brain. 2015; 7:47.
3. Hoyt W F, Pont M E. Pseudopapilledema: anomalous elevation of optic disk: pitfalls in diagnosis and management. JAMA. 1962; 181:191-196.
4. Freund P, Margolin E. Pseudopapilledema. In. StatPearls [Internet]. Treasure Island, Fla.: StatPearls Publishing; 2019. Available at: www.ncbi.nlm.nih.gov/books/NBK538291/.
5. Chiang J, Wong E, Whatham A, Hennessy M, Kalloniatis M, Zangerl B. The usefulness of multimodal imaging for differentiating pseudopapilloedema and true swelling of the optic nerve head: a review and case series. Clin Exp Optom. 2015; 98:12-24.
6. Chang M Y, Velez F G, Demer J L, Bonelli L, Quiros P A, Arnold A C, Sadun A A, Pineles S L. Accuracy of diagnostic imaging modalities for classifying pediatric eyes as papilledema versus pseudopapilledema. Ophthalmology. 2017; 124:1839-1848.
7. Carter S B, Pistilli M, Livingston K G, Gold D R, Volpe N J, Shindler K S, Liu G T, Tamhankar M A. The role of orbital ultrasonography in distinguishing papilledema from pseudopapilledema. Eye. 2014; 28:1425.
8. Saenz R, Cheng H, Prager T C, Frishman L J, Tang R A. Use of A-scan ultrasound and optical coherence tomography to differentiate papilledema from pseudopapilledema. Optom Vis Sci. 2017; 94:1081-1089.
9. Johnson L N, Diehl M L, Hamm C W, Sommerville D N, Petroski G F. Differentiating optic disc edema from optic nerve head drusen on optical coherence tomography. Arch Ophthalmol. 2009; 127:45-49.
10. Carta A, Mora P, Aldigeri R, Gozzi F, Favilla S, Tedesco S, Calzetti G, Farci R, Barboni P, Bianchi-Marzoli S, Fossarello M. Optical coherence tomography is a useful tool in the differentiation between true edema and pseudoedema of the optic disc. PLoS One. 2018; 13:e0208145.
11. Martinez M R, Ophir A. Optical coherence tomography as an adjunctive tool for diagnosing papilledema in young patients. J Pediatr Ophthalmol Strabismus. 2011; 48:174-181.
12. Sarac O, Tasci Y Y, Gurdal C, Can I. Differentiation of optic disc edema from optic nerve head drusen with spectral-domain optical coherence tomography. J Neuroophthal. 2012; 32:207-211.
13. Pardon L P, Cheng H, Tang R A, Saenz R, Frishman L J, Patel N B. Custom optical coherence tomography parameters for distinguishing papilledema from pseudopapilledema. Optom Vis Sci. 2019; 96:599-608.
14. Bassi S T, Mohana K P. Optical coherence tomography in papilledema and pseudopapilledema with and without optic nerve head drusen. Indian J Ophthalmol. 2014; 62:1146-1151.
15. Lee K M, Woo S J, Hwang J M. Differentiation of optic nerve head drusen and optic disc edema with spectral-domain optical coherence tomography. Ophthalmology. 2011; 118:971-977.
16. Kulkarni K M, Pasol J, Rosa P R, Lam B L. Differentiating mild papilledema and buried optic nerve head drusen using spectral domain optical coherence tomography. Ophthalmology. 2014; 121:959-963.
17. Fard M A, Sahraiyan A, Jalili J, Hejazi M, Suwan Y, Ritch R, Subramanian P S. Optical coherence tomography angiography in papilledema compared with pseudopapilledema. Invest Ophthalmol Vis Sci. 2019; 60:168-175.
18. Pineles S L, Arnold A C. Fluorescein angiographic identification of optic disc drusen with and without optic disc edema. J Neuroophthalmol. 2012; 32:17.
19. Jindahra P, Petrie A, Plant G T. The time course of retrograde trans-synaptic degeneration following occipital lobe damage in humans. Brain. 2012; 135:534-541.
20. Budenz D L, Anderson D R, Varma R, Schuman J, Cantor L, Savell J, Greenfield D S, Patella V M, Quigley H A, Tielsch J. Determinants of normal retinal nerve fiber layer thickness measured by Stratus OCT. Ophthalmology. 2007; 114:1046-1052.
21. Youden W J. Index for rating diagnostic tests. Cancer. 1950; 3:32-35.
22. Perkins N J, Schisterman E F. The inconsistency of “optimal” cutpoints obtained using two criteria based on the receiver operating characteristic curve. Am J Epidemiol. 2006; 163:670-675.
23. Liu X. Classification accuracy and cut point selection. Stat Med. 2012; 31:2676-2686.
24. Gampa A, Vangipuram G, Shirazi Z, Moss H E. Quantitative association between peripapillary Bruch's membrane shape and intracranial pressure. Invest Ophthalmol Vis Sci. 2017; 58:2739-2745.
25. Sibony P, Kupersmith M J, Honkanen R, Rohlf F J, Torab-Parhiz A. Effects of lowering cerebrospinal fluid pressure on the shape of the peripapillary retina in intracranial hypertension. Invest Ophthalmol Vis Sci. 2014; 55:8223-8231.
26. Kupersmith M J, Sibony P, Mandel G, Durbin M, Kardon R H. Optical coherence tomography of the swollen optic nerve head: deformation of the peripapillary retinal pigment epithelium layer in papilledema. Invest Ophthalmol Vis Sci. 2011; 52:6558-6564.
27. Kardon R Optical coherence tomography in papilledema: what am I missing? J Neuroophthalmol. 2014; 34(suppl):S10-S17.
28. Malmqvist L, Wegener M, Sander B A, Hamann S. Peripapillary Retinal Nerve Fiber Layer Thickness Corresponds to Drusen Location and Extent of Visual Field Defects in Superficial and Buried Optic Disc Drusen. J Neuroophthalmol. 2016; 36(1):41-45.
29. Fard M A, Sahraiyan A, Jalili J, et al. Optical Coherence Tomography Angiography in Papilledema Compared With Pseudopapilledema. Invest Ophthalmol Vis Sci. 2019; 60(1):168-175.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method for determining a magnitude and variability of retinal nerve fiber layer (RNFL) thickness of an elevated optic nerve in a subject, comprising:
(a) detecting in the subject a retinal nerve fiber layer (RNFL) thickness for each of twelve clock-hour positions;
(b) calculating a magnitude value (M), where M=(Y₁+Y₂+Y₃+Y₄+Y₅+Y₆+Y₇+Y₈+Y₉+Y₁₀+Y₁₁+Y₁₂)/12;
(c) calculating a variability value (V), where V=(Y₁−Y₁₂)+(Y₂−Y₁)+(Y₃−Y₂)+(Y₄−Y₃)+(Y₅−Y₄)+(Y₆−Y₅)+(Y₇−Y₆)+(Y₈−Y₇)+(Y₉−Y₈)+(Y₁₀−Y₉)+(Y₁₁−Y₁₀)+(Y₁₂−Y₁₁); and
(d) calculating a magnitude-variability value (X), where X=(B₁*M)+(B₂*V), B₁ is a magnitude coefficient, and B₂ is a variability coefficient;
wherein Yₙ is the RNFL thickness for the n clock hour position.

2. The method of claim 1, and further comprising detecting the RNFL thickness for each of twelve clock-hour positions using optical coherence tomography (OCT).

3. The method of claim 2, wherein B₁ is 0.08-0.12; and B₂ is 0.045-0.055.

4. The method of claim 2, wherein B₁ is about 0.11; and B₂ is about 0.049.

5. The method of claim 2, wherein B₁ is about 0.1007; and B₂ is about 0.0493.

6. The method of claim 1, wherein B₁ is 0.08-0.12; and B₂ is 0.045-0.055.

7. The method of claim 1, wherein B₁ is about 0.11; and B₂ is about 0.049.

8. The method of claim 1, wherein B₁ is about 0.1007; and B₂ is about 0.0493.

9. A method for differentiating between papilledema and pseudopapilledema, comprising:
(a) identifying a subject with an elevated optic nerve;
(b) detecting in the subject a retinal nerve fiber layer (BNFL) thickness at each of twelve clock-hour positions using optical coherence tomography (OCT); and
(c) identifying the subject as having pseudopapilledema when X is less than 13.2, and declining to identify the subject has having pseudopapilledema when X is greater than 13.2;
wherein
X=(B₁*M)+(B₂*V);
B₁ is 0.08-0.12;
B₂ is 0.045-0.055;
M=(Y₁+Y₂+Y₃+Y₄+Y₅+Y₆+Y₇+Y₈+Y₉+Y₁₀+Y₁₁+Y₁₂)/12; and
V=(Y₁−Y₁₂)+(Y₂−Y₁)+(Y₃−Y₂)+(Y₄−Y₃)+(Y₅−Y₄)+(Y₆−Y₅)+(Y₇−Y₆)+(Y₈−Y₇)+(Y₉−Y₈)+(Y₁₀−Y₉)+(Y₁₁−Y₁₀)+(Y₁₂−Y₁₁);
wherein Yₙ is the BNFL thickness for the n clock hour position.

10. The method of claim 9, wherein B₁ is about 0.11; and B₂ is about 0.049.

11. The method of claim 9, wherein B₁ is about 0.1007; and B₂ is about 0.0493.

12. The method of claim 11, and further comprising identifying the subject as having papilledema when X is greater than 13.2

13. The method of claim 12, and further comprising administering treatment for papilledema when X is greater than 13.2.

14. The method of claim 11, and further comprising identifying the subject as having papilledema when X is greater than 14.8.

15. The method of claim 14, and further comprising administering treatment for papilledema when X is greater than 14.8.

16. The method of claim 9, and further comprising identifying the subject as having papilledema when X is greater than 13.2

17. The method of claim 16, and further comprising administering treatment for papilledema when X is greater than 13.2.

18. The method of claim 9, and further comprising identifying the subject as having papilledema when X is greater than 14.8.

19. The method of claim 18, and further comprising administering treatment for papilledema when X is greater than 14.8.
